(19) United States
(12) Patent Application Publication
(10) Pub. No.: US 2023/0052021 A1
(43) Pub. Date: Feb. 16, 2023

(54) DISPLAY DEVICE AND DISPLAY METHOD

(71) Applicant: Nippon Telegraph and Telephone Corporation, Tokyo (JP)

(72) Inventors: Arinobu NIIJIMA, Musashino-shi, Tokyo (JP); Toki TAKEDA, Musashino-shi, Tokyo (JP); Takafumi MUKOUCHI, Musashino-shi, Tokyo (JP); Takashi SATO, Musashino-shi, Tokyo (JP)

(21) Appl. No.: 17/797,515

(22) PCT Filed: Feb. 6, 2020
(86) PCT No.: PCT/JP2020/004592

§ 371 Date: Aug. 4, 2022

(51) Int. Cl. A61F 7/00

(52) U.S. Cl. CPC A61F 7/007; B65D 2215/00

(57) ABSTRACT

A presentation device includes a stimulation presentation unit that presents temperature stimulation at a predetermined temperature and a temperature control unit that sets the temperature of the stimulation presentation unit. The stimulation presentation unit has a size that causes a person to feel temperature stimulation when the stimulation presentation unit touches a first portion and the person not to feel the temperature stimulation when the stimulation presentation unit touches a second portion that is less perceptible than the first portion. The temperature control unit sets the temperature of the stimulation presentation unit so that the stimulation presentation unit presents pain due to temperature stimulation when the stimulation presentation unit touches the first portion and does not present the pain due to temperature stimulation even when the stimulation presentation unit touches the second portion.

### TECHNICAL FIELD

The present invention relates to a presentation device and a presentation method.

### BACKGROUND ART

Accidental ingestion by children in daily life has become a problem. For example, there is a method of preventing accidental ingestion by children by attaching a bitter component to the target of accidental ingestion by children such as a toy. However, when the sense of taste of children is not sufficiently developed, the children may prefer bitterness. Even if a bitter component is attached to the target of accidental ingestion, the effect of preventing accidental ingestion is limited.

In addition, it is known that lips are more sensitive to temperature than fingertips and that the smaller the presentation area of temperature stimulation, the more difficult to perceive temperature (NPL 1 and 2).

### CITATION LIST

### Non Patent Literature

[NPL 1] Lynette A. Jones and Hsin-Ni Ho, “Warm or cool, large or small? The challenge of thermal displays”, IEEE TRANSACTIONS ON HAPTICS, VOL. 1, NO. 1, JANUARY-JUNE 2008
[NPL 2] JOSEPH C. STEVENS and KENNETH K. CHOO, “Temperature sensitivity of the body surface over the life span”, Somatosensory & Motor Research 1998; 15(1): 13-28

### SUMMARY OF THE INVENTION

### Technical Problem

Since prevention of accidental ingestion by children by use of bitterness is limited, a more appropriate method of preventing accidental ingestion by children is required.

For example, it is considered that a sensor that detects contact with a mouth and an actuator that causes pain at the time of detection of contact with a mouth are attached to the target of accidental ingestion. However, this method may need the cost of providing the sensor and the actuator. In addition, the sensor may be required to have a high recognition accuracy to detect contact with a mouth.

The present invention addresses the situations described above with an object of providing a technique for appropriately preventing accidental ingestion by children.

### Means for Solving the Problem

According to an aspect of the present invention, there is provided a presentation device including: a stimulation presentation unit that presents temperature stimulation at a predetermined temperature; and a temperature control unit that sets a temperature of the stimulation presentation unit, in which the stimulation presentation unit has a size that causes a person to feel the temperature stimulation when the stimulation presentation unit touches a first portion and the person not to feel the temperature stimulation when the stimulation presentation unit touches a second portion that is less perceptible than the first portion, and the temperature control unit sets the temperature of the stimulation presentation unit so that the stimulation presentation unit presents pain due to the temperature stimulation when the stimulation presentation unit touches the first portion and does not present the pain due to the temperature stimulation even when the stimulation presentation unit touches the second portion.

According to an aspect of the present invention, there is provided a presentation device including: a stimulation presentation unit alternately provided with a warm sensation presentation unit that presents warm stimulation and a cold sensation presentation unit that presents cold stimulation; and a temperature control unit that sets a temperature of the stimulation presentation unit, in which the temperature control unit sets a temperature of the warm sensation presentation unit and a temperature of the cold sensation presentation unit so that the stimulation presentation unit presents pain due to thermal grill illusion when the stimulation presentation unit touches a first portion and does not present the pain due to thermal grill illusion when the stimulation presentation unit touches a second portion that is less perceptible than the first portion.

According to an aspect of the present invention, there is provided a presentation method used by a presentation device including a stimulation presentation unit that presents temperature stimulation at a predetermined temperature and a temperature control unit that sets a temperature of the stimulation presentation unit, the stimulation presentation unit having a size that causes a person to feel temperature stimulation when the stimulation presentation unit touches a first portion and the person not to feel the temperature stimulation when the stimulation presentation unit touches a second portion that is less perceptible than the first portion, the presentation method including: the step of causing the temperature control unit to set the temperature of the stimulation presentation unit so that the stimulation presentation unit presents pain due to temperature stimulation when the stimulation presentation unit touches the first portion and does not present the pain due to temperature stimulation even when the stimulation presentation unit touches the second portion; and the step of causing the stimulation presentation unit to present temperature stimulation at a set temperature.

According to an aspect of the present invention, there is provided a presentation method used by a presentation device including a stimulation presentation unit alternately provided with a warm sensation presentation unit that presents warm stimulation and a cold sensation presentation unit that presents cold stimulation and a temperature control unit that sets a temperature of the stimulation presentation unit, the presentation method including: the step of causing the temperature control unit to set a temperature of the warm sensation presentation unit and a temperature of the cold sensation presentation unit so that the stimulation presentation unit presents pain due to thermal grill illusion when the stimulation presentation unit touches a first portion and does not present the pain due to thermal grill illusion when the stimulation presentation unit touches a second portion that is less perceptible than the first portion; and the step of causing the stimulation presentation unit to present temperature stimulation at a set temperature.

### Effects of the Invention

According to the present invention, it is possible to provide a technique capable of appropriately preventing accidental ingestion by children.

### BRIEF DESCRIPTION OF DRAWINGS

FIG. 1 is a figure illustrating the functional block of a presentation device according to an embodiment of the present invention.

FIG. 2 is a figure illustrating the hardware structure of the presentation device according to the embodiment of the present invention.

FIG. 3 is a figure illustrating an example of the structure of a stimulation presentation unit according to the first example and the operation thereof.

FIG. 4 is a figure illustrating an example of the structure of the stimulation presentation unit according to a second example and the operation thereof.

FIG. 5 is a figure illustrating an example of the structure of a stimulation presentation unit according to a third example and the operation thereof.

### DESCRIPTION OF EMBODIMENTS

An embodiment of the present invention will be described with reference to the drawings. In the description of the drawings, the same components are designated by the same reference numeral and the description thereof will be omitted.

(Presentation Device)

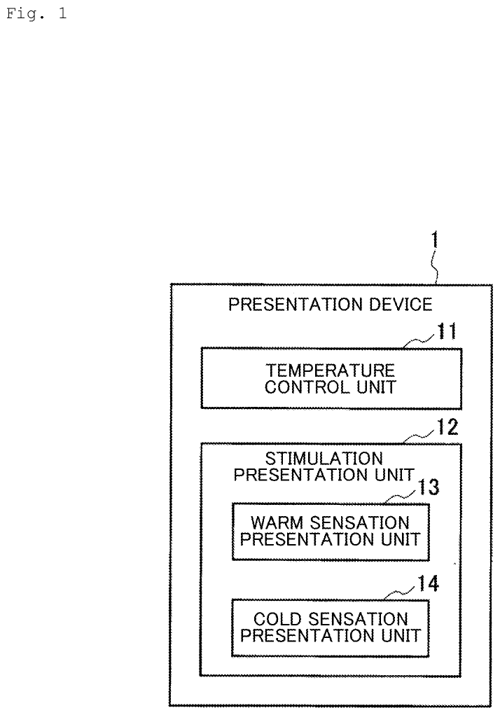

As illustrated in FIG. 1, a presentation device 1 includes a temperature control unit 11 and a stimulation presentation unit 12.

Temperature control unit 11 sets and controls the temperature of the stimulation presentation unit.

The stimulation presentation unit 12 presents the temperature stimulation at the temperature set by the temperature control unit 11. When the stimulation presentation unit 12 touches a portion that perceives temperature, such as lips or a hand of a person, the stimulation presentation unit 12 presents the person with temperature stimulation by temperature.

The stimulation presentation unit 12 includes a warm sensation presentation unit 13 and a cold sensation presentation unit 14. The warm sensation presentation unit 13 presents a warm sensation to the person through the portion touched by the warm sensation presentation unit 13. The cold sensation presentation unit 14 presents a cold sensation to the person through the portion touched by the cold sensation presentation unit 14.

In the embodiment of the present invention, the presentation device 1 is formed so as to present stimulation when touching a temperature-sensitive portion such as lips and not to present stimulation when touching a temperature-insensitive portion such as a fingertip.

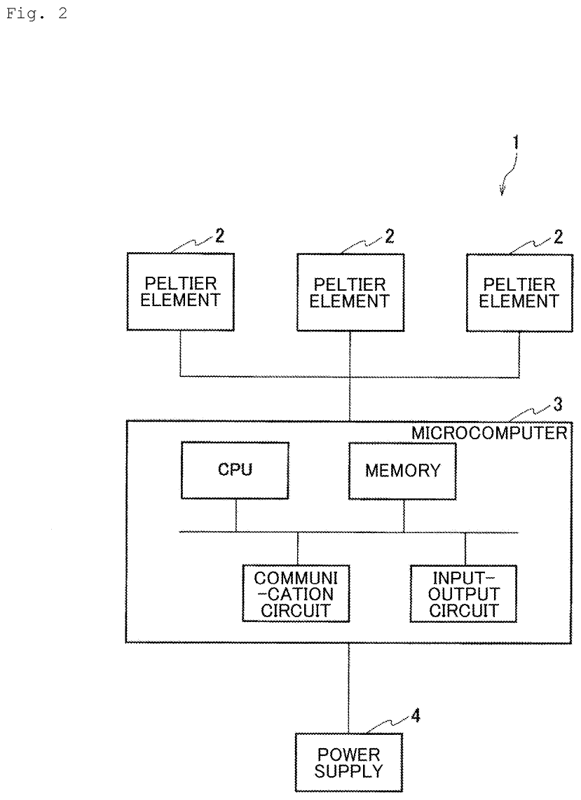

The hardware structure indicating an implementation example of the presentation device 1 will be described with reference to FIG. 2. The presentation device 1 includes Peltier elements 2, a microcomputer 3 (micro-controller), and a power supply 4 as hardware.

The Peltier elements 2 function as the stimulation presentation unit 12 in FIG. 1. The Peltier element 2 is one of thermoelectric elements (semiconductor thermoelectric elements) using the Peltier effect. The Peltier element 2 is used as an example of the thermoelectric element in the embodiment of the present invention, but another thermoelectric element can be used to present temperature stimulation. In the embodiment of the present invention, the presentation device 1 has one or more Peltier elements 2.

The Peltier element 2 has a p-n junction portion that changes in temperature when receiving an electric current. The Peltier element 2 is provided so that the temperature of the p-n junction portion is transferred to the person who touches the presentation device 1. The Peltier element 2 may be provided so that, for example, the p-n junction portion is exposed to the surface of the presentation device 1 or an any member such as a ceramic substrate may be provided on the outside of the Peltier element 2. The person receives temperature stimulation when touching the p-n junction portion or the member covering the p-n junction portion. It should be noted that the size of the Peltier element 2 is selected as appropriate according to the use method described later.

The microcomputer 3 is a computer that includes a CPU, a memory, a communication circuit, an input-output circuit, and the like and functions as the temperature control unit 11 in FIG. 1. The microcomputer 3 is connected to the Peltier elements 2 via lead wires (not illustrated) or the like. The microcomputer 3 causes the Peltier elements 2 to present temperature stimulation by applying an electric current to the Peltier elements 2. The microcomputer 3 controls the driving of the Peltier elements 2.

The microcomputer 3 can change the temperature of the p-n junction portions of the Peltier elements by applying an electric current to the Peltier elements 2 and can present temperature stimulation to the portion touched by the presentation device 1. In addition, by controlling the current values (the direction of current and the magnitude of current) applied to the Peltier elements 2, the microcomputer 3 can select cold stimulation or warm stimulation as the temperature stimulation and can also control the temperature (the strength of temperature stimulation) to be presented.

The power supply 4 supplies electric power to the microcomputer 3.

It should be noted that, in the presentation device 1, the functions of temperature control unit 11 and the like can be achieved by causing the CPU to read and execute predetermined programs. The programs of presentation device 1 can be stored on a computer readable recording medium such as a HDD, a SSD, a universal serial bus (USB) memory, a compact disc (CD), or a digital versatile disc (DVD), or can be delivered via a network.

To show the target of accidental ingestion to a child, different information needs to be indicated to the child depending on whether the target of accidental ingestion touches a mouth or lips or the target of accidental ingestion touches other portions such as a hand. Specifically, when accidental ingestion of a toy is prevented, it is possible to prevent a child from eating the toy while playing with the toy by causing the child not to feel pain when the hand of the child touches the toy and the child to feel pain when the lips of the child touch the toy.

As described in NPL 1 and 2, it is known that lips are more sensitive to temperature than fingertips and that the smaller the presentation area of temperature stimulation, the more difficult to perceive temperature.

Accordingly, the presentation device 1 according to the embodiment of the present invention has the stimulation presentation unit 12 with a size that causes a temperature-sensitive portion such as lips to feel temperature stimulation and temperature-insensitive portion such as a fingertip not to feel temperature stimulation. When the fingertip of the child touches the stimulation presentation unit 12 of the presentation device 1, the child does not feel anything and can continue to touch the target of accidental ingestion. In contrast, when the lips of the child touch the stimulation presentation unit 12, the child feels a pain so as to prevent accidental ingestion.

Next, three implementation methods using the presentation device 1 will be described. In these implementation methods, the presentation device 1 presents pain to the first portion and does not present pain to a second portion different from the first portion. The first portion and the second portion are the portions in which the person feels temperature. The first portion is the portion such as lips that is sensitive to temperature. The second portion is a fingertip or the like that is less perceptible than the first portion.

In the embodiment of the present invention, “perception” refers to sensitivity to temperature stimulation or pain. “Less perceptible” means less sensitive to temperature stimulation or insensitive to temperature stimulation presented in a narrow area, but sensitive to temperature stimulation presented in a wide area. In the embodiment of the present invention, an example of a more perceptible portion is lips and an example of a less perceptible portion is a fingertip.

### First Example

In the first example, the presentation device 1 has at least one stimulation presentation unit 12.

When the stimulation presentation unit 12 touches the first portion directly or indirectly, the first portion feels temperature and the second portion does not feel temperature. The stimulation presentation unit 12 has a size that causes the person to feel temperature stimulation when the stimulation presentation unit touches the first portion and the person not to feel temperature stimulation when the stimulation presentation unit touches the second portion. The size of the portion of the stimulation presentation unit 12 touched by the person is, for example, approximately 10 mm².

The temperature control unit 11 sets the temperature of the stimulation presentation unit 12 so that the stimulation presentation unit 12 presents pain due to temperature stimulation when the stimulation presentation unit 12 touches the first portion and does not present pain due to temperature stimulation even when the stimulation presentation unit 12 touches the second portion. The temperature control unit 11 sets the temperature of the stimulation presentation unit 12 so that the person who touches the stimulation presentation unit 12 feels pain. When the stimulation presentation unit 12 presents pain due to warm stimulation, the temperature control unit 11 controls the stimulation presentation unit 12 so that the temperature of the stimulation presentation unit 12 becomes a value (for example, 45° C. or higher) higher than that of the human body. When the stimulation presentation unit 12 presents pain due to cold stimulation, the temperature control unit 11 controls the stimulation presentation unit 12 so that the temperature of the stimulation presentation unit 12 becomes a value (for example, 15° C. or lower) lower than that of the human body.

It should be noted that the size and the temperature of the stimulation presentation unit 12 may be changed as appropriate.

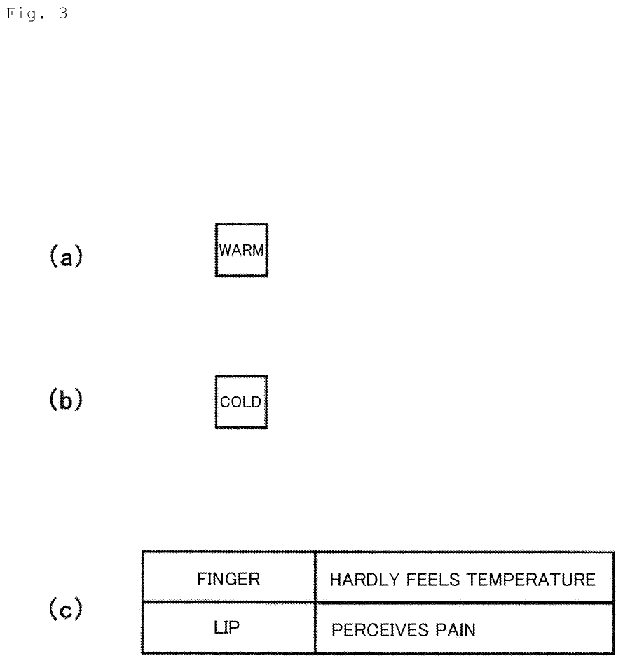

In the first example, the presentation device 1 presents warm stimulation or cold stimulation as illustrated in FIG. 3(a) or 3(b). In addition, the size of the stimulation presentation unit 12 is formed so that the person who touches the stimulation presentation unit 12 feels temperature stimulation with the relatively sensitive lips and does not feel temperature stimulation with the relatively insensitive finger, whereby the person hardly feels temperature when touching with a finger and the person perceives pain due to warm sensation or cold sensation when touching with lips as illustrated in FIG. 3(b).

As described above, the stimulation presentation unit 12 of the presentation device 1 according to the first example has a size that causes the person to feel temperature stimulation in the first portion and the person not to feel temperature stimulation in the second portion and presents the temperature at which the person feels pain. Accordingly, the presentation device 1 presents pain when the presentation device 1 touches the first portion and does not present pain when the presentation device 1 touches the second portion.

As described above, the presentation device 1 according to the first example can cause the person to feel pain when the presentation device 1 touches the portion sensitive to temperature and the person not to feel pain when the presentation device 1 touches the portion insensitive to temperature.

### Second Example

In the second example, a plurality of Peltier elements 2 are provided as the plurality of stimulation presentation units 12 in the presentation device 1. The plurality of stimulation presentation units 12 are disposed adjacent to each other.

The plurality of stimulation presentation units 12 include stimulation presenting units that present a temperature at which pain is felt and stimulation presenting units that present a temperature (specifically, a temperature close to the temperature of the portion to be touched) at which the temperature is hardly felt. Here, the number of stimulation presentation units that present pain due to temperature stimulation among the plurality of stimulation presentation units is smaller than the number of stimulation presenting units that do not present pain due to temperature stimulation among the plurality of stimulation presentation units. The temperature of the stimulation presentation unit 12 that presents pain due to temperature stimulation is outside the range of the body temperature, specifically, not lower than 45° C. and not higher than 15° C. The temperature of the stimulation presentation unit 12 that does not present pain due to temperature stimulation is approximately the body temperature, for example, approximately 20° C.

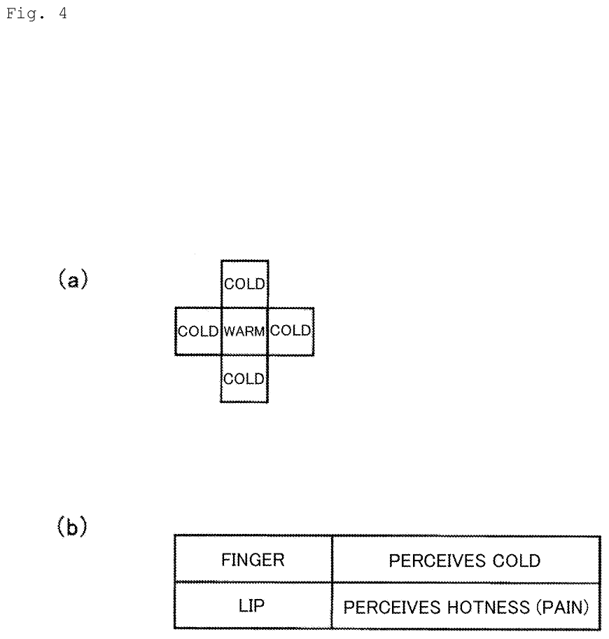

The plurality of stimulation presentation units 12 in the second example are formed as illustrated in, for example, FIG. 4(a). The stimulation presentation unit 12 that presents a temperature at which pain due to warm stimulation is felt is formed in the center, and a plurality of stimulation presentation units 12 that present cold stimulation at a temperature at which pain is not felt are formed therearound.

Since the first portion is sensitive to temperature and can perceive temperature even in a small area as illustrated in FIG. 4(b), the first portion can perceive the temperatures of the plurality of stimulation presentation units 12. In the example illustrated in FIG. 4(a), when the stimulation presentation unit 12 that presents a temperature enough to present pain touches a temperature-sensitive portion, the person feels pain.

In contrast, the second portion is insensitive to temperature, cannot perceive temperature in a small area, and perceives the temperature obtained by integrating the temperatures of the plurality of stimulation presentation units 12 by spatial weighting. In the example illustrated in FIG. 4(a), there is the stimulation presentation unit 12 that presents a temperature enough to present pain, but there are more stimulation presentation units 12 that present temperatures not enough to present pain. Accordingly, when such stimulation presentation units 12 touch a temperature-insensitive portion, the person does not feel pain.

It should be noted that the size and the disposition of each of the plurality of stimulation presentation units 12 are determined so that the person can feel the temperature of each of the stimulation presentation units 12 when touching with the temperature-sensitive portion and the person can feel the temperature calculated by spatial weighting of the temperature of the stimulation presentation units 12 when touching with the temperature-insensitive portion.

As described above, the presentation device 1 according to the second example has the plurality of stimulation presentation units 12 in which some of the stimulation presentation units 12 present a temperature causing pain and others present a temperature not causing pain. As a result, when the plurality of stimulation presentation units 12 touch the temperature-sensitive portion, the person feels pain due to the stimulation presentation units 12 that present the temperature causing pain. In contrast, when the number of stimulation presentation units 12 that present the temperature not causing pain is more than the number of stimulation presentation units 12 that present the temperature causing pain, when the plurality of stimulation presentation units 12 touch the temperature-insensitive portion, even if the plurality of stimulation presentation units 12 include the stimulation presentation units 12 that present the temperature causing pain, the person does not feel pain because the temperatures of the stimulation presentation units 12 are averaged by spatial weighting.

As described above, the presentation device 1 according to the second example cause the person to feel pain when the presentation device 1 touches the temperature-sensitive portion and cannot cause the person to feel pain when the presentation device 1 touches the temperature-insensitive portion.

### Third Example

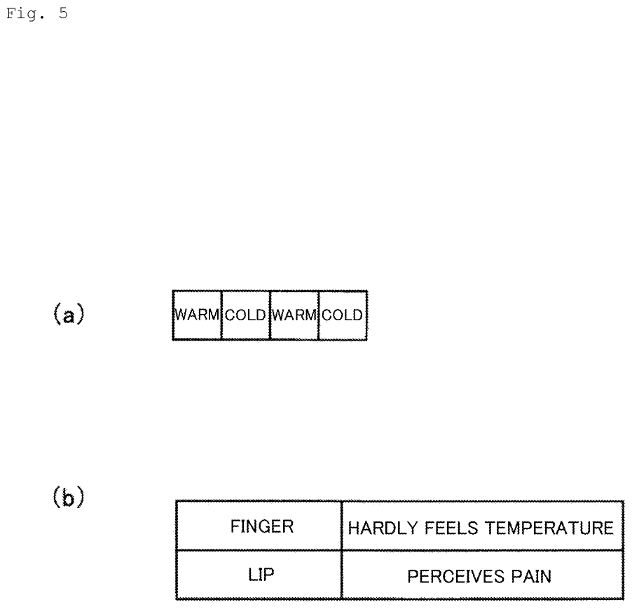

In the third example, the stimulation presentation unit 12 of the presentation device 1 has one or more warm sensation presentation units 13 that present warm stimulation and one or more cold sensation presentation units 14 that present cold stimulation. In the third example, the warm sensation presentation unit 13 and the cold sensation presentation unit 14 are alternately provided as illustrated in FIG. 5(a).

The stimulation presentation unit 12, the warm sensation presentation unit 13, and the cold sensation presentation unit 14 have sizes that cause the person to feel pain due to thermal grill illusion when these presentation units touch the first portion and cause the person not to feel pain due to thermal grill illusion when these presentation units touch the second portion.

The temperature control unit 11 sets the temperatures of the warm sensation presentation unit 13 and the cold sensation presentation unit 14 so as to present pain due to thermal grill illusion when the stimulation presentation unit 12 touches the first portion and not to present pain due to thermal grill illusion when the stimulation presentation unit 12 touches the second portion.

As illustrated in FIG. 5(b), in the third example, when touching with the temperature-sensitive first portion, the warm stimulation and the cold stimulation are received individually, thermal grill illusion is produced and the person feels pain. In contrast, when touching with the temperature-insensitive second portion, the warm stimulation and the cold stimulation are averaged by spatial weighting, thermal grill illusion is not produced, and the person hardly feels temperature.

The temperature of the warm sensation presentation unit 13 at which thermal grill illusion is produced in the temperature-sensitive portion is, for example, approximately 40° C. and the temperature of the cold sensation presentation unit 14 is, for example, approximately 20° C.

It should be noted that the sizes and the dispositions of the warm sensation presentation unit 13 and the cold sensation presentation unit 14 are determined so that pain due to thermal grill illusion is felt when touching the temperature-sensitive portion and the temperature calculated by spatial weighting of the temperatures of the warm sensation presentation unit 13 and the cold sensation presentation unit 14 is felt when touching the temperature-insensitive portion.

As described above, the presentation device 1 according to the third example can cause the person to feel pain when the presentation device 1 touches temperature-sensitive portion and can cause the person not to feel pain when the presentation device 1 touches the temperature-insensitive portion.

It should be noted that the present invention is not limited to the embodiment described above and many modifications can be made within the scope of the concept.

### REFERENCE SIGNS LIST

1 Presentation device
2 Peltier element
11 Temperature control unit
12 Stimulation presentation unit
13 Warm sensation presentation unit
14 Cold sensation presentation unit

1. A presentation device comprising:
a stimulation presentation unit, implemented with one or more processors, configured to present temperature stimulation at a predetermined temperature; and
a temperature control unit, implemented with one or more processors, configured to set a temperature of the stimulation presentation unit,
wherein the stimulation presentation unit has a size that causes a person to experience the temperature stimulation when the stimulation presentation unit touches a first portion of the person and the person does not to experience the temperature stimulation when the stimulation presentation unit touches a second portion of the person that is less perceptible than the first portion of the person, and
the temperature control unit sets the temperature of the stimulation presentation unit so that the stimulation presentation unit presents pain due to the temperature stimulation when the stimulation presentation unit touches the first portion of the person and does not present the pain due to the temperature stimulation even when the stimulation presentation unit touches the second portion of the person.

2. The presentation device according to claim 1,
wherein a plurality of stimulation presentation units are provided, the stimulation presentation unit being one of the plurality of stimulation presentation units.

3. The presentation device according to claim 2,
wherein the number of stimulation presentation units that present the pain due to the temperature stimulation among the plurality of stimulation presentation units is less than a number of the plurality of stimulation presentation units that do not present the pain due to the temperature stimulation among the plurality of stimulation presentation units.

4. A presentation device comprising:
a stimulation presentation unit, implemented with one or more processors, comprising a warm sensation presentation unit configured to present warm stimulation and a cold sensation presentation unit configured to present cold stimulation; and
a temperature control unit, implemented with one or more processors, configured to set a temperature of the stimulation presentation unit,
wherein the temperature control unit sets a temperature of the warm sensation presentation unit and a temperature of the cold sensation presentation unit so that the stimulation presentation unit presents pain due to thermal grill illusion when the stimulation presentation unit touches a first portion of a person and does not present the pain due to thermal grill illusion when the stimulation presentation unit touches a second portion of the person that is less perceptible than the first portion.

5. A presentation method used by a presentation device including a stimulation presentation unit configured to present temperature stimulation at a predetermined temperature and a temperature control unit configured to set a temperature of the stimulation presentation unit, the stimulation presentation unit comprising a size to cause a person to feel the temperature stimulation when the stimulation presentation unit touches a first portion of the person and the person not to feel the temperature stimulation when the stimulation presentation unit touches a second portion of the person that is less perceptible than the first portion, the presentation method comprising:
setting, by the temperature control unit, the temperature of the stimulation presentation unit so that the stimulation presentation unit presents pain due to the temperature stimulation when the stimulation presentation unit touches the first portion of the person and does not present the pain due to the temperature stimulation even when the stimulation presentation unit touches the second portion of the person; and
presenting, by the stimulation presentation unit, temperature stimulation at a set temperature.

6. (canceled)
